(19) United States
(12) Patent Application Publication
(10) Pub. No.: US 2025/0367098 A1
(43) Pub. Date: Dec. 4, 2025

(54) SUNSCREEN COMPOSITION FOR LACTOBACILLUS AND S. EPIDERMIDIS PROTECTION ON SKIN

(71) Applicant: DSM IP Assets B.V., Maastricht (NL)

(72) Inventors: Rolf SCHUETZ, Kaiseraugst (CH); Jürgen Herbert VOLLHARDT, Kaiseraugst (CH)

(21) Appl. No.: 18/873,973

(22) PCT Filed: Jun. 7, 2023
(86) PCT No.: PCT/EP2023/065165

§ 371 Date: Dec. 11, 2024

(30) Foreign Application Priority Data
Jun. 15, 2022 (EP) 22179304.5

(51) Int. Cl. A61K 8/49; A61K 8/35; A61K 8/40; A61K 31/121; A61K 31/277; A61K 31/53; A61P 17/16; A61Q 17/04; A61Q 19/00

(52) U.S. Cl. CPC A61K 8/4966; A61K 8/35; A61K 8/40; A61K 31/121; A61K 31/277; A61K 31/53; A61P 17/16; A61Q 17/04; A61Q 19/00

(57) ABSTRACT

The present invention relates to the use of selected UV filters selected from the group consisting of octocrylene, ethyl hexyltriazone and butyl methoxydibenzoylmethane for the concomitant protection of skin bacteria of the genus Lactobacillus such as e.g., Lactobacillus crispatus (L. crispatus) and S. epidermidis against UV radiation.

The present invention relates to the use of selected UV filters selected from the group consisting of octocrylene, ethyl hexyltriazone and butyl methoxydibenzoylmethane for the concomitant protection of skin bacteria of the genus Lactobacillus such as e.g., Lactobacillus crispatus (L. crispatus) and S. epidermidis against UV radiation.

Endogenous inhabitants of healthy skin are lactobacilli (family Lactobacilliae) such as L. crispatus. They have been described to play a beneficial role in the skin habitat, where they exert multifactorial local mechanisms of action against pathogens and inflammation. On one hand, lactobacilli have been shown to directly compete with skin pathogens through adhesion inhibition, production of antimicrobial metabolites, and by influencing pathogen metabolism. The competitive anti-pathogenic action of lactobacilli has already been described mechanistically for common different skin pathogens, such as Staphylococcus aureus, Cutibacterium acnes, and Candida albicans. On the other hand, lactobacilli also have an immunomodulatory capacity associated with a reduction in excessive skin inflammation. Their influence on the immune system is mediated by bacterial metabolites and cell wall-associated or excreted microbe-associated molecular patterns (MAMPs). In addition, lactobacilli can also enhance skin barrier function, e.g., when being disrupted because of infection or inflammatory skin diseases.

It is well known that UV-light destroys microbes such as bacteria.

Thus, there is an ongoing need of selectively protecting beneficial bacteria from UV-radiation such as in particular of bacteria of the genus Lactobacillus. In addition, a protection of S. epidermidis, another is also desirable.

Accordingly, the inventors developed a targeted in vitro liquid model in 96-well microplate format to screen for UV protective actives (“UV filters”) on bacterial survival upon UV irradiation. The effects of selected UV irradiation conditions tested on microbial population of Lactobacillus crispatus (L. crispatus) and Staphylococcus epidermidis (S. epidermidis) in the presence of UV filter samples and combinations thereof were evaluated with the evolution of the residual microbial population after 4 h. The inhibition of bacterial growth, as consequence of UV irradiation, was compared to that of the same strain without UV irradiation and was expressed in percentage of survival (%).

Surprisingly, it was now found that certain UV-filter substances are particularly suitable to protect bacteria of the genus Lactobacillus such as in particular L. crispatus as well as S. epidermidis against the detrimental effects of UV-radiation by significantly and selectively improving their survival rate after irradiation with UV-light.

Thus, in a first embodiment, the present invention is directed to an use of one or more UV-filters selected from the group consisting of octocrylene, butyl methoxydibenzoylmethane and ethylhexyl triazone oxide for the concomitant protection of bacteria of the genus Lactobacillus, preferably L. crispatus and S. epidermidis from the detrimental effects of UV radiation.

Because of increase in number, i.e. an increase in the abundance of bacteria of the genus Lactobacillus as well as of S. epidermidis after UV-radiation, metabolites secreted by said bacteria are also likely to increase.

The protection of skin bacteria of the genus Lactobacillus such as in particular of L. crispatus according to the present invention is characterized by an increase in the survival rate of bacteria of the genus Lactobacillus such as in particular of L. crispatus by at least 100% over control after exposure to UV-radiation (@50 SED).

S. epidermidis is concomitantly protected from UV-radiation as well. Said protection is characterized by an increase in the survival rate of S. epidermidis by at least 20% over control after exposure to UV-radiation (@15 SED).

A particularly preferred UV-filter according to the present invention is octocrylene as well as a mixture of ethylhexyl triazone, butylmethoxydibenzoyl methane and bis-ethylhexyloxyphenol methoxyphenyl triazine as they lead to a particularly good survival rate of skin bacteria of the genus Lactobacillus such as in particular of L. crispatus while still efficiently protecting S. epidermidis.

The most preferred Lactobacillus in all embodiments of the present invention is Lactobacillus crispatus CIP 103604.

The most preferred Staphylococcus epidermidis in all embodiments of the present invention is Staphylococcus epidermidis ATCC 12228.

In all embodiments of the present invention, a particularly preferred bacteria of the genus Lactobacillus species is L. crispatus.

The term UV-radiation (synonymously used with UV-light) as used herein refers to radiation in the UVA-UVB range of 280 to 400 nm.

In all embodiments of the present invention, the amount of ethylhexyl triazone (also known as 2,4,6-tris [anilino (p-carbo-2′-ethyl-1′-hexyloxy)]-1,3,5-triazine (EHT)) used according to the present invention is preferably selected in the range from 0.1 wt.-% to 10 wt.-%, more preferably in the range from 0.5 wt.-% to 7.5 wt. %, most preferably in the range from 1 wt.-% to 5 wt.-%.

In all embodiments of the present invention, the amount of butyl methoxydibenzoylmethane (also known as avobenzone, respectively 3-(4-tert-Butylphenyl)-1-(4-methoxyphenyl) propane-1,3-dione (BMDBM)) used according to the present invention is preferably selected in the range from 0.1 wt.-% to 10 wt.-%, more preferably in the range from 0.5 wt.-% to 7.5 wt.-%, most preferably in the range from 1 wt.-% to 5 wt.-%.

In all embodiments of the present invention, the amount of octocrylene (also known as 2-ethylhexyl 2-cyano-3,3-diphenylprop-2-enoate respectively ethylhexyl 2-cyano-3,3-diphenylacrylate (OC)) used according to the present invention is preferably selected in the range from 0.1 wt.-% to 10 wt.-%, more preferably in the range from 0.5 wt.-% to 7.5 wt.-%, most preferably in the range from 1 wt.-% to 5 wt.-%.

In a particular embodiment, a combination of octocrylene, ethylhexylsalicylate, bis-ethylhexyloxyphenol methoxyphenyl triazine, and butyl methoxydibenzoylmethane, more preferably in a total amount of 5-20 wt.-%, preferably in the range of 7.5 to 15, most preferably in the range from 7.5 to 12 wt.-% is used.

The UV filters are preferably contained in an appropriate carrier such as in a composition comprising an oil selected from the group consisting of

The exact amount of carrier will depend upon the actual level of the UV filters and any other optional ingredients that one of ordinary skill in the art would classify as distinct from the carrier (e.g., other active ingredients).

In an advantageous embodiment, the sunscreen compositions according to the present invention comprise from 50 wt.-% to 99 wt.-%, preferably from 60 wt.-% to 98 wt.-%, more preferably from 70 wt.-% to 98 wt.-%, such as in particular from 80 wt.-% to 95 wt.-% of a carrier, based on the total weight of the sunscreen composition.

In a particular advantageous embodiment, the carrier consists furthermore of at least 40 wt.-%, more preferably of at least 50 wt.-%, most preferably of at least 55 wt.-% of water, such as in particular of 55 wt.-% to 90 wt.-% of water.

In particular, the sunscreen composition according to the present invention are cosmetic or pharmaceutical compositions, preferably cosmetic (non-therapeutic) compositions.

In one embodiment, the sunscreen compositions according to the present invention are applied to mammalian keratinous tissue such as in particular to human skin.

The term “cosmetic composition” as used in the present application refers to cosmetic compositions as defined under the heading “Kosmetika” in Römpp Lexikon Chemie, 10th edition 1997, Georg Thieme Verlag Stuttgart, New York as well as to cosmetic compositions as disclosed in A. Domsch, “Cosmetic Compositions”, Verlag für chemische Industrie (ed. H. Ziolkowsky), 4th edition, 1992.

The sunscreen compositions according to the invention are also known as light-protective preparations (sun care products, sunscreens), such as sun protection milks, sun protection lotions, sun protection creams, sun protection oils, sun blocks or tropical's or day care creams with a SPF (sun protection factor). Of particular interest are sun protection creams, sun protection lotions, sun protection milks and sun protection preparations.

The compositions of the invention (including the carrier) may comprise conventional adjuvants and additives, such as preservatives/antioxidants, fatty substances/oils, organic solvents, silicones, thickeners, softeners, emulsifiers, antifoaming agents, aesthetic components such as fragrances, surfactants, fillers, anionic, cationic, nonionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such compositions.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self-tanning, soothing, as well as agents to improve elasticity and skin barrier and/or further UV-filter substances and carriers and/or excipients or diluents conventionally used in sunscreen compositions.

If nothing else is stated, the excipients, additives, diluents, etc. mentioned in the following are suitable for sunscreen compositions according to the present invention. The necessary amounts of the cosmetic and dermatological adjuvants and additives can, based on the desired product, easily be determined by the skilled person.

The additional ingredients can either be added to the oily phase, the aqueous phase or separately as deemed appropriate. The mode of addition can easily be adapted by a person skilled in the art.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

The sunscreen compositions according to the present invention may be in the form of a suspension or dispersion in solvents or fatty substances, or alternatively in the form of an emulsion or micro emulsion (in particular of oil-in-water (O/W—) or water-in-oil (W/O—) type, silicone-in-water (Si/W—) or water-in-silicone (W/Si—) type, PIT-emulsion, multiple emulsion (e.g. oil-in-water-in oil (O/W/O—) or water-in-oil-in-water (W/O/W—) type), pickering emulsion, hydrogel, alcoholic gel, lipogel, one- or multiphase solution or vesicular dispersion or other usual forms, which can also be applied by pens, as masks or as sprays.

The sunscreen compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art and illustrated in the examples.

In one advantageous embodiment, O/W emulsifier is a phosphate ester emulsifier. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C₆₋₁₀ Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4

Phosphate. A particular phosphate ester emulsifier according to the invention is potassium cetyl phosphate e.g. commercially available as Amphisol® K at DSM Nutritional Products Ltd Kaiseraugst.

Further suitable O/W emulsifiers according to the present invention encompass PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, glycerylstearatcitrate, glycerylstearate (self-emulsifying), stearic acid, salts of stearic acid, polyglyceryl-3-methylglycosedistearate. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

Another particular suitable class of O/W emulsifiers are non-ionic self-emulsifying system derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

Further suitable are commercially available polymeric emulsifiers such as hydrophobically modified polyacrylic acid such as Acrylates/C10-30 Alkyl Acrylate Crosspolymers which are commercially available under the tradename Pemulen® TR-1 and TR-2 by Noveon.

Another class of particularly suitable emulsifiers are polyglycerol esters or diesters of fatty acids also called polyglyceryl ester/diester (i.e. a polymer in which fatty acid(s) is/are bound by esterification with polyglycerine), such as e.g. commercially available at Evonik as Isolan GPS [INCI Name Polyglyceryl-4 Diisostearate/Polyhydroxystearate/Sebacate (i.e. diester of a mixture of isostearic, polyhydroxystearic and sebacic acids with Polyglycerin-4)] or Dehymuls PGPH available at Cognis (INCI Polyglyceryl-2 Dipolyhydroxystearate).

Also suitable are polyalkylenglycolether such as Brij 72 (Polyoxyethylen(2)stearylether) or Brij 721 (Polyoxyethylene (21) Stearyl Ether e.g. available at Croda.

Particularly advantageous O/W emulsifiers according to the present invention are one or more of Polyglyceryl-3 Methylglucose Distearate, Lauryl Glucoside (and) Polyglyceryl-2 Dipolyhydroxystearate, Glyceryl Sterate Citrate, Sodium Cetearyl Sulfate, Cetearyl Glucoside; Polyglyceryl-6 Stearate (and) Polyglyceryl-6 Behenate, Cetearyl Olivate (and) Sorbitan Olivate, Arachidyl Alcohol (and) Behenyl Alcohol (and) Arachidyl Glucosides, Cetearyl Alcohol (and) Coco-Glucoside, Coco-Glucoside (and) Coconut Alcohol, PEG-100 Stearate (and) Glyceryl Stearate, Sodium Stearoyl Glutamate, Steareth-20, Steareth-21, Steareth-25, Steareth-2, Ceteareth-25 and Ceteareth-6 (all listed by their INCI names).

Most preferred O/W emulsifiers according to the present invention are one or more of Ceteareth-6 (optionally in admixture with stearyl alcohol) and Ceteareth-25.

The at least one O/W respectively Si/W emulsifier is preferably used in an amount of 0.5 to 10 wt.-% such as in particular in the range of 0.5 to 5 wt.-% such as most in particular in the range of 0.5 to 4 wt.-%, based on the total weight of the composition.

Suitable W/O- or W/Si-emulsifiers according to the present invention are polyglyceryl-2-dipolyhydroxystearat, PEG-30 dipolyhydroxystearat, cetyl dimethicone copolyol, polyglyceryl-3 diisostearate polyglycerol esters of oleic/isostearic acid, polyglyceryl-6 hexaricinolate, polyglyceryl-4-oleate, polygylceryl-4 oleate/PEG-8 propylene glycol cocoate, magnesium stearate, sodium stearate, potassium laurate, potassium ricinoleate, sodium cocoate, sodium tallowate, potassium castorate, sodium oleate, and mixtures thereof. Further suitable W/Si-emulsifiers are Lauryl Polyglyceryl-3 Polydimethylsiloxyethyl Dimethicone and/or PEG-9 Polydimethylsiloxyethyl Dimethicone and/or Cetyl PEG/PPG-10/1 Dimethicone and/or PEG-12 Dimethicone Crosspolymer and/or PEG/PPG-18/18 Dimethicone. A particularly suitable W/O emulsifier to be used in the compositions according to the present invention is PEG-30 dipolyhydroxystearat. The at least one W/O emulsifier is preferably used in an amount of about 0.001 to 10 wt.-%, more preferably in an amount of 0.2 to 7 wt.-% with respect to the total weigh of the composition.

The sunscreen compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 7 wt.-%, such as most in particular in the range of 1 to 5 wt.-%, based on the total weight of the composition. Particular suitable co-surfactants are selected from the list of alkyl alcohols such as cetyl alcohol (Lorol C16, Lanette 16), cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof.

In a particular advantageous embodiment according to the present invention the emulsifier is selected from the group of Ceteareth-6 and/or Ceteareth-25 and the co-surfactant is selected from the group of behenyl alcohol, cetyl alcohol, cetearyl alcohol and/or stearyl alcohol.

The compositions in form of O/W emulsions according to the invention can be provided, for example, in all the formulation forms for O/W emulsions, for example in the form of serum, milk or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

In one advantageous aspect of the invention, the sunscreen compositions according to the present invention do not contain (i.e. are free of) 3-(4-methylbenzylidene)-camphor, and 2-hydroxy-4-methoxybenzophenone (INCI: Oxybenzone).

In another advantageous aspect of the invention, the sunscreen compositions according to the present invention further comprise one or more of dibutyl adipate, dicaprylyl carbonate, stearyl alcohol, C12-C15 alkylbenzoate, Caprylyl Carbonate, Capric/Caprylic Triglyceride as well as mixtures thereof, preferably dibutyl adipate, stearyl adipate and/or dicaprylyl carbonate.

In another advantageous aspect of the invention, the sunscreen compositions according to the present invention further comprise ethanol, more preferably in an amount of 0.5 wt.-% to 10 wt.-%, more preferably of 3 wt.-% to 8 wt.-%, based on the total weight of the sunscreen composition.

In a still further advantageous aspect of the invention, the sunscreen compositions of the present invention further comprise a preservative and/or a preservative booster, preferably selected from the group consisting of phenoxyethanol, ethylhexylglycerin, hexylglycerin, glyceryl caprylate, caprylyl glycol, 1,2-hexanediol, propanediol, propylene glycol, p-hydroxyacetophenone as well as mixtures thereof, most preferably selected from the group of phenoxyethanol and ethylhexylglycerine as well as mixtures thereof. When present, the preservative respectively the preservative booster is preferably used in an amount of 0.01 to 2 wt.-%, more preferably in an amount of 0.05 wt.-% to 1.5 wt.-%, most preferably in an amount of 0.1 wt.-% to 1.0 wt.-% such as in an amount of 0.1 wt.-% to 0.5 wt.-%, based on the total weight of the composition.

In another advantageous aspect, the sunscreen compositions according to the present invention are free of any parabenes, benzethoniumchlorid, piroctone olamine, lauroylarginat, methylisothiazolinon, chlormethylisothiazolinon, bronopol, benzalkoniumchloride, formaldeh releasing compounds, salicylic acid, triclosan, DMDM hydantoin, chlorphenesin and IPBC (lodopropinylbutyl carbamate).

In a still further advantageous aspect, the sunscreen compositions of the present invention further comprise one or more of glycerol or polyhydroxystearic acid as well as mixtures thereof.

In yet a still further advantageous aspect, the sunscreen compositions of the present invention further comprise a thickening agent, preferably a gum such as xanthan gum or Caesalpina spinosa gum or a polyacrylate such as polyacrylate crosspolymer-6 as well as mixtures thereof.

The sunscreen compositions according to the invention in general have a pH in the range of 3 to 10, preferably a pH in the range of 4 to 8 and most preferably a pH in the range of 4 to 7. The pH can easily be adjusted as desired with suitable acids such as e.g. citric acid or bases such as NaOH according to standard methods in the art.

The sunscreen compositions according to the invention may further contain one or more emollients which soothe and soften the skin. As an example, the emollient may be dicaprylyl carbonate or dibutyl adipate. Further emollients are silicone (dimethicone, cyclomethicone), vegetable oils (grape seed, sesame seed, jojoba, etc.), butters (cocoa butter, shea butter), alcohols (stearyl alcohol, cetyl alcohol), and petrolatum derivatives (petroleum jelly, mineral oil).

In yet another aspect, the sunscreen composition of the invention may comprise potassium cetylphosphate as emulsifier and/or one or more of dibutyl adipate, dicaprylyl carbonate and stearoyl alcohol.

In yet another aspect, the sunscreen composition of the invention may comprise acrylate copolymers such as acrylate/C₁₀₋₃₀ alkyl acrylate crosspolymer, even more preferably in amounts of 0.05 wt.-% to 1 wt.-%, more preferably 0.1 wt.-% to 0.5 wt.-%, based on the total weight of the sunscreen composition.

In another aspect, the sunscreen composition of the invention may comprise one or more fragrances selected from limonene, citral, linalool, alpha-isomethylionone, geraniol, citronellol, 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic diester, cinnamal, amyl salicylate, alpha-amylcinnamaldehyde, alpha-methylionone, butylphenylmethylpropional, cinnamal, amylcinnamyl alcohol, anise alcohol, benzoin, benzyl alcohol, benzyl benzoate, benzyl benzyl oil, cinnamate, salicylate, bergamot bitter orange oil, butylphenylmethylpropional, cardamom oil, cedrol, cinnamal, cinnamyl alcohol, citronellyl methylcrotonate, citrus oil, coumarin, diethyl succinate, ethyllinalool, eugenol, Evernia furfuracea extract, Evernia prunastri extract, farnesol, guaiacwood oil, hexylcinnamal, hexyl salicylate, hydroxycitronellal, lavender oil, lemon oil, linalyl acetate, mandarin oil, menthyl PCA, methyl heptenone, nutmeg oil, rosemary oil, sweet orange oil, terpineol, tonkabean oil, triethyl citrate, vanillin.

In another aspect, the sunscreen composition of the invention may contain at least one salt of 2-phenylbenzimidazole-5-sulfonic acid.

In another aspect, the composition may have an SPF of at least 20, preferably of at least 30, such as for example a SPF of 20 to 50 or 30 to 50.

The sunscreen compositions of the invention manage with a surprisingly small total amount of UV filters.

It is advantageous in accordance with the invention if the sunscreen composition is present in the form of an emulsion or dispersion, preferably in the form of an emulsion, and more preferably in the form of an O/W emulsion.

Where the sunscreen composition of the invention is in the form of an O/W emulsion, the preparation advantageously comprises one or more O/W emulsifiers selected from glyceryl stearate citrate, glyceryl stearate (self-emulsifying), stearic acid, stearate salts, polyglyceryl-3-methylglycose distearate, sodium cetearylsulfate, potassium cetylphosphate, polyglyceryl-10 stearate, and sodium stearylglutamate.

Advantageously in accordance with the invention, these O/W emulsifiers of the invention may be present in the preparation in a concentration of 0.001 wt.-% to 10 wt % and preferably in a concentration of 0.1 wt.-% to 7 wt %, based on the total weight of the preparation.

It is preferred in accordance with the invention if the preparation comprises potassium cetyl phosphate as emulsifier.

It is further advantageous in accordance with the invention if the preparation comprises cetyl alcohol, stearyl alcohol and/or glycerylstearate.

It is of advantage in accordance with the invention if the preparation of the invention is free from polyethylene glycol, polyethylene glycol ethers, and polyethylene glycol esters (so-called PEG derivatives).

The preparation of the invention may advantageously comprise moisturizers. Moisturizers are compounds or mixtures of compounds which give cosmetic preparations the quality, after application to or distribution on the skin surface, of reducing the loss of moisture of the stratum corneum (also called transepidermal water loss (TEWL)) and/or of positively influencing the hydration of the stratum corneum.

Non-limiting examples of advantageous moisturizers for use in the present invention include glycerol, lactic acid and/or lactates, especially sodium lactate, butylene glycol, propylene glycol, biosaccharide gum-1, Glycine soya, ethylhexyloxyglycerol, pyrrolidonecarboxylic acid, and urea. Of further advantage, in particular, is the use of polymeric moisturizers from the group of the polysaccharides which are water-soluble and/or swellable in water and/or gellable with the aid of water. Especially advantageous, for example, are hyaluronic acid, chitosan and/or a fucose-rich polysaccharide which is registered in Chemical Abstracts under the registry number 178463-23-5 and is available, for example, under the Fucogel®1000 name from the company SOLABIA S.A. Moisturizers may also be used advantageously as active antiwrinkle ingredients for protection from changes to the skin of the kind occurring in skin aging, for example.

The cosmetic preparations of the invention may further comprise advantageously, although not mandatorily, fillers which have the effect, for example, of further improving the sensorial and cosmetic properties of the formulations and evoking or intensifying a velvety or silky skin sensation, for example. Advantageous fillers in the sense of the present invention are starch and starch derivatives (such as tapioca starch, distarch phosphate, aluminum or sodium starch octenylsuccinate, and the like, for example), pigments which have neither primarily UV filter effect nor coloring effect (such as boron nitride, etc., for example) and/or Aerosils® (CAS No. 7631-86-9) and/or talc and/or polyethylene, nylon, and silica dimethyl silylate.

Advantageous embodiments of the preparation of the present invention also include those wherein the preparation comprises one or more oils selected from butylene glycol dicaprylate/dicaprate, phenethyl benzoate, C₁₂₋₁₅ alkyl benzoate, dibutyl adipate; diisopropyl sebacates, dicaprylyl carbonate, di-C₁₂₋₁₃ alkyl tartrates, butyloctyl salicylates, diethylhexyl syringylidene malonates, hydrogenated castor oil dimerates, triheptanoin, C₁₂₋₁₃ alkyl lactates, C₁₆₋₁₇ alkyl benzoates, propylheptyl caprylates, caprylic/capric triglycerides, diethylhexyl 2,6-naphthalates, octyldodecanol, ethylhexyl cocoates.

It is preferred in accordance with the invention if the preparation comprises dibutyl adipate, dicaprylyl carbonate and/or C_{12-C15} alkyl benzoate.

The water phase of the preparations of the invention may advantageously comprise customary cosmetic auxiliaries, such as, for example, alcohols, particularly those of low C number, preferably ethanol and/or isopropanol, or polyols of low C number, and also ethers thereof, preferably propylene glycol, glycerol, electrolytes, self-tanning agents, and also, in particular, one or more thickeners, which may be advantageously selected from the group of silicon dioxide, aluminum silicates, polysaccharides and/or derivatives thereof, e.g., hyaluronic acid, xanthan gum, hydroxypropylmethylcellulose, particularly advantageously from the group of polyacrylates, preferably a polyacrylate from the group referred to as Carbopols, examples being carbopols of types 980, 981, 1382, 2984, and 5984, in each case individually or in combination. Further thickeners advantageous in accordance with the invention are those having the INCI designation Acrylates/C10-30 Alkyl Acrylate Crosspolymer (e.g., Pemulen TR 1, Pemulen TR 2, Carbopol 1328 from NOVEON) and also Aristoflex AVC (INCI: Ammonium Acryloyldimethyltaurate/VP Copolymer) as well as Simugel NS (INCI: Hydroxyethyl Acrylate/Sodium Acryloyldimethyl Taurate Copolymer & Squalane & Polysorbate 60).

It is preferred here in accordance with the invention if the preparation comprises xanthan gum, crosslinked acrylate/C_{10-C30} alkyl acrylate polymer, Hydroxyethyl Acrylate/Sodium Acryloyldimethyl Taurate Copolymer & Squalane & Polysorbate 60 and/or vinylpyrrolidone/hexadecene copolymer.

An amount of glycerol of at least 2.5 wt %, based on the total weight of the preparation, is particularly advantageous in accordance with the invention.

It is advantageous in accordance with the invention if the preparation comprises one or more alkanediols from the group 1,2-pentanediol, 1,2-hexanediol, 1,2-octanediol, 1,2-decanediol, 2-methyl-1,3-propanediol.

It is advantageous in accordance with the invention if the preparation of the invention comprises ethanol, phenoxyethanol and/or ethylhexylglycerin.

Advantageously in accordance with the invention, the preparation of the invention comprises film formers. Film formers in the sense of the present invention are substances of various kinds and are characterized by the following properties: When a film former is dissolved in water or other suitable solvents, and when the solution is then applied to the skin, the film former, following evaporation of the solvent, forms a film which serves essentially to fix the UV-filters on the skin and to so increase the SPF and/or water resistance of the product.

It is especially advantageous to select the film formers from the group of the polymers based on polyvinylpyrrolidone (PVP)

Particular preference is given to copolymers of vinylpyrrolidone, as for example the PVP hexadecene copolymer and the PVP eicosene copolymer, which are available under the trade names Antaron V216 and Antaron V220 from GAF Chemicals Corporation.

Likewise advantageous are further polymeric film formers, such as, for example, sodium polystyrene sulfonate, which is available under the trade name Flexan 130 from National Starch and Chemical Corp., and/or polyisobutene, available from Rewo under the trade name Rewopal PIB1000. Examples of further suitable polymers are polyacrylamides (Seppigel 305), polyvinyl alcohols, PVP, PVP/VA copolymers, polyglycols, acrylate/octylacrylamide copolymer (Dermacryl 79) Likewise advantageous is the use of hydrogenated castor oil dimer dilinoleate (CAS 646054-62-8, INCI Hydrogenated Castor Oil Dimer Dilinoleate), which can be acquired from Kokyu Alcohol Kogyo under the name Risocast DA-H, or else PPG-3 benzyl ether myristate (CAS 403517-45-3), which can be acquired under trade name Crodamol STS from Croda Chemicals.

The following examples are provided to further illustrate the compositions and effects of the present invention. These examples are illustrative only and are not intended to limit the scope of the invention in any way.

### EXAMPLES

### In Vitro UV Model

The microbial strains used in this study are listed below and their cultures are reconstituted according to the direction of the supplier collection center of the reference strain, CRBIP (Institut Pasteur Collection, France) or ATCC (American Type Culture Collection, USA):
Lactobacillus crispatus CIP 103604
Staphylococcus epidermidis ATCC 12228

The 96-well plate was filled with the culture media (140 μL) and a calibrated inoculum (20 μL) of each bacterial strain was added to reach 1.106 colony forming units per milliliter (cfu/mL) final concentration. After contamination, the sample was added (40 μL; 20% v/v) in triplicate and was thoroughly mixed to ensure a homogeneous distribution. Then, the plate was submitted to UV stress through an UV irradiator system (BioSun, Vilber Lourmat, France) according to the UV doses presented Table 1 below. The specific UV dose selected was between 10 and 49% survival of the microbial population.

TABLE 1
Selection of the complying UV irradiation conditions for each microbial
strain. The energy ration between UVA and UVB was 1:19.

| | | | Survival after |
|---|---|---|---|
| Strains | SED (units) | UVA + UVB (mJ/cm²) | 4 h (%) |
| S. epidermidis | 15 | 150 | ±* |
| L. crispatus | 50 | 500 | − |

±*: Survival of the microbial population between 21% and 49%;
−: Survival of the microbial population equal to or less than 20%.

For the microbial population counts, aliquots of the contaminated samples of L. crispatus and S. epidermidis were serially diluted (4 decimal dilutions followed by 8 binary dilutions, in triplicate) in culture media on 96-wells plates with triphenyltetrazolium. Then, the plates were incubated at 37° C.±2,5° C. during 48 h taking into account the respiratory type of each strain. After 48 h at the optimal growing conditions, the last dilution showing a pink color (triphenyltetrazolium) or turbidity indicated the contamination rate and allowed the determination of the decrease rate of microbial population (most probable number MPN method). Controls performed during this experiment were the culture medium with bacteria incubated without UV stress. The microbial population counts were expressed in cfu/mL and were the average of experiment done in triplicate.

The use level of the single UV filter and combinations in the test formulations was calculated to obtain a predicted in vitro SPF value of 5.75+/−0.15 and 23.45+/−0.65, respectively (Table 2). The UV filters were mixed in the two oils, Tegosoft XC (55%) and Miglyol 829 Eco (ad100%) and the emulsifier Tween 80 (5%).

The effect of selected UV irradiation conditions on the survival of microbial population of L. crispatus and S. epidermidis in the presence of the tested samples 1-10 at 20% v/v, was compared with the corresponding strain in the absence of samples 1-10 and without exposure to UV irradiation (control). The results after the 4-hour incubation were compared to the population of bacteria incubated without UV stress (determination of bacterial population survival) and were expressed in percentage of survival. The relative percentage of protection (%) was calculated from the difference between the survival percentages of samples and vehicle control (with UV irradiation) and divided to the survival percentage of the vehicle control. Consequently, the survival of each microbial population and the relative protection by the UV filter(s) were calculated and are summarized in the Table 2.

TABLE 2
Test formulations of UV filter(s) and ingredients (* mixed
with 55% Tegosoft XC, ad100% Miglyol 829 Eco, 5% Tween
80) in weight-%. Results of the survival of each microbial
population compared to untreated and non-irradiated control
was calculated in %. In brackets: the relative protection
by the UV filter(s) vs. vehicle control is given in %.

| | UV filters | | L. crispatus | | S. epidermidis | |
|---|---|---|---|---|---|---|
| # | * | Wt-% | survival (protection [%]) | | | |
| Inv. 1 | OC | 5 | 50 | (+300) | 63 | (+59) |
| Inv. 2 | BMDBM | 5 | 25 | (+100) | 63 | (+59) |
| Inv. 3 | EHT | 2.3 | 25 | (+100) | 63 | (+59) |
| Inv. 5 | EHT/BMDBM/ | 3/3/3 | 31 | (+152) | 50 | (+26) |
| | BEMT | | | | | |
| Ref. 1 | EHS | 10 | 20 | (+59) | 79 | (+100) |
| Ref. 2 | BEMT | 2.3 | 20 | (+59) | 79 | (+100) |
| Ref. 3 | TiO₂/SiO₂/D | 3 | 20 | (+59) | 50 | (+26) |
| Control | Vehicle control | | 12.5 | (0) | 40 | (0) |

| Abbreviations | INCI Name |
|---|---|
| OC | Octocrylene |
| BMDBM | Butyl methoxydibenzoylmethane |
| ZnO Tecs | Zinc oxide, Triethoxycaprilylsilane |
| EHT | Ethylhexyl Triazone |
| BEMT | Bis-Ethylhexyloxyphenol Methoxyphenyl Triazine |
| EHS | Ethylhexyl Salicylate |
| TiO₂/SiO₂/D | Titanium Dioxide, Silica, Dimethicone |

1. Use of one or more UV-filters selected from the group consisting of octocrylene, butyl methoxydibenzoylmethane and ethylhexyl triazone oxide for the concomitant protection of bacteria of the genus Lactobacillus, preferably L. crispatus and S. epidermidis from the detrimental effects of UV radiation.

2. The use according to claim 1, wherein the protection of the bacteria of the genus Lactobacillus is characterized by an increase in the survival rate by at least 100% and the protection of S. epidermidis is characterized by an increase in the survival rate by 2% over control after exposure to UV-radiation.

3. The use according to claim 1, wherein the skin bacteria of the genus of Lactobacillus is L. crispatus.

4. The use according to claim 1, wherein the skin bacteria of the genus of Lactobacillus is L. crispatus CIP 103604.

5. The use according to claim 1, wherein the skin bacteria S. epidermidis is Staphylococcus epidermidis ATCC 12228

6. The use according to claim 1, wherein ethylhexyl triazone is used in an amount selected in the range from 0.1 wt.-% to 10 wt.-%, preferably in the range from 0.5 wt.-% to 7.5 wt.-%, most preferably in the range from 1 wt.-% to 5 wt.-%.

7. The use according to claim 1, wherein butyl methoxydibenzoylmethane is used in an amount selected in the range from 0.1 wt.-% to 10 wt.-%, preferably in the range from 0.5 wt.-% to 7.5 wt.-%, most preferably in the range from 1 wt.-% to 5 wt.-%.

8. The use according to claim 1, wherein octocrylene is used in an amount selected in the range from 0.1 wt.-% to 10 wt.-%, preferably in the range from 0.5 wt.-% to 7.5 wt.-%, most preferably in the range from 1 wt.-% to 5 wt.-%.
